(19) United States
(12) Patent Application Publication
(10) Pub. No.: US 2021/0116337 A1
(43) Pub. Date: Apr. 22, 2021

(54) LIQUID SAMPLE PROCESSING METHOD AND LIQUID SAMPLE PROCESSING APPARATUS

(71) Applicant: SHIMADZU CORPORATION, Kyoto (JP)

(72) Inventor: Hiroshi IWATA, Kyoto-shi (JP)

(21) Appl. No.: 17/069,043

(22) Filed: Oct. 13, 2020

(30) Foreign Application Priority Data
Oct. 18, 2019 (JP) 2019-191185

(51) Int. Cl. G01N 1/40; B01L 3/00; B01D 61/14; B01D 71/04

(52) U.S. Cl. CPC G01N 1/4077; B01L 3/502; B01D 61/147; B01D 2311/04; G01N 2001/4088; B01L 2200/0647; B01L 2300/0681; B01D 71/04

(57) ABSTRACT

The present disclosure provides a liquid sample processing method capable of efficiently removing blood cells. The liquid sample processing method is configured to remove blood cells out of a liquid sample containing blood cells, and includes filtering the liquid sample by allowing measurement objects to pass through the filter faster than the blood cells, and filtering the liquid sample after the measurement objects are made to pass through the filter faster than the blood cells so as to remove the blood cells.

### BACKGROUND OF THE INVENTION

### Field of the Invention

The present disclosure relates to a liquid sample processing method and a liquid sample processing apparatus.

### Description of the Background Art

In analyzing a blood sample, there is known a method of purifying microorganisms such as bacteria and nucleic acids which serve as measurement objects by removing blood cell components which may affect the accuracy of measurement. For example, Martin Christner, Holger Rohde, Manuel Wolters, Ingo Sobottka, Karl Wegscheider, and Martin Aepfelbacher reported, on Journal of Clinical Microbiology, 48(5): 1584-1591 (2010), a method of purifying bacteria from cultured blood by performing centrifugation and filtration stepwise.

### SUMMARY OF THE INVENTION

The method mentioned above requires stepwise operations, which is troublesome and time consuming.

The present disclosure has been accomplished in view of the aforementioned problem, and an object thereof is to provide a liquid sample processing method and a liquid sample processing apparatus capable of easily removing blood cells out of a liquid sample containing blood cells.

A liquid sample processing method according to the present disclosure includes performing, by using a first filter, a first filtration on a liquid sample, the liquid sample containing blood cells and measurement objects smaller than the blood cells, the first filter having a first filtration resistance against the measurement objects and a second filtration resistance against the blood cells, and the first filtration resistance being smaller than the second filtration resistance, so that the measurement objects generally pass through the first filter faster than the blood cells, and performing, by using a second filter, a second filtration on a filtrate from the first filter, the filtrate containing the measurement objects, while the blood cells are retained on the first filter, to selectively permeate the measurement objects by size so as to obtain a final filtrate in which the blood cells are removed out of the liquid sample.

A liquid sample processing apparatus of the present disclosure includes a container which houses a liquid sample containing blood cells and measurement objects smaller than the blood cells, a first filter which is disposed in the container and configured to permeate the blood cells and the measurement objects smaller than the blood cells and allow the measurement objects to permeate faster than the blood cells, and a second filter which is disposed in the container on the downstream of the first filter and configured to selectively capture the blood cells by size and selectively permeate the measurement objects by size.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 is a diagram schematically illustrating an overall configuration of a recovery apparatus including a processing apparatus according to a first embodiment;

FIG. 2 is a diagram schematically illustrating the configuration of the processing apparatus according to the first embodiment;

FIG. 3 is a diagram schematically illustrating the movement of blood cells and measurement objects when a liquid sample is filtered by a first filter and a second filter in the processing apparatus according to the first embodiment;

FIG. 4 is a diagram schematically illustrating an overall configuration of a recovery apparatus including a processing apparatus according to a second embodiment; and

FIG. 5 is a diagram schematically illustrating an overall configuration of a recovery apparatus including a processing apparatus according to a third embodiment.

### DESCRIPTION OF THE PREFERRED EMBODIMENTS

Hereinafter, embodiments of the present disclosure will be described in detail with reference to the drawings. In the drawings, the same or corresponding portions are denoted by the same reference numerals, and the description thereof will not be repeated.

A liquid sample processing apparatus according to the present disclosure is an apparatus for processing a liquid sample such as blood containing blood cells, and is used, for example, in a pretreatment before measuring a substance (measurement objects) having a size smaller than blood cells in blood. Further, the liquid sample processing apparatus according to the present disclosure is configured to filter a liquid sample containing blood cell components such as blood by full amount filtration. Furthermore, the liquid sample processing apparatus according to the present disclosure is preferably disposable for the purpose of obtaining the measurement objects. The measurement objects are smaller than blood cells (especially red blood cells), and may include, for example, microorganisms, nucleic acids, and proteins in plasma. Microorganisms may include, for example, bacteria such as germs and fungi, viruses and molds. Hereinafter, the liquid sample processing apparatus is simply referred to as the processing apparatus where necessary.

### First Embodiment

The liquid sample processing apparatus according to a first embodiment is a blood cell removing apparatus having a function of removing blood cells, and is incorporated in a recovery apparatus for recovering measurement objects from a liquid sample.

<Configuration of Recovery Apparatus>

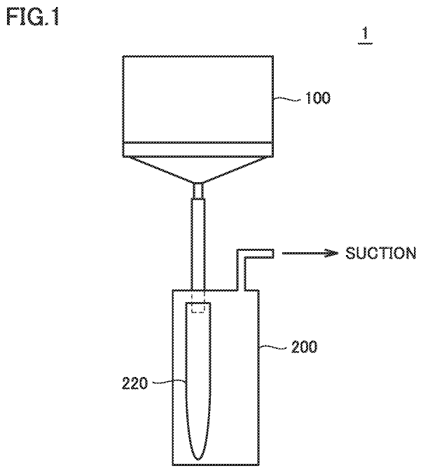

FIG. 1 is a diagram schematically illustrating an overall configuration of a recovery apparatus including a processing apparatus according to a first embodiment. With reference to FIG. 1, a recovery apparatus 1 includes a processing apparatus 100, a suction box 200, and a recovery container 220 installed in the suction box 200.

The processing apparatus 100 is a filter unit. An outlet (secondary side) of the processing apparatus 100 is connected to the suction box 200. The suction box 200 is connected to a vacuum pump via a pipe (both not shown).

The vacuum pump is driven to reduce a pressure inside the suction box 200. After a liquid sample such as whole blood is filled in the processing apparatus 100, when the pressure inside the suction box 200 is reduced, the liquid sample in the processing apparatus 100 is filtered by suction filtration. The filtrate is recovered in the recovery container 220.

The filtrate recovered in the recovery container 220 is filled in a measurement apparatus arbitrarily selected in accordance with the measurement objects. The filtrate recovered in the recovery container 220 may be subjected to another pretreatment and then filled in the measurement apparatus.

<Configuration of Processing Apparatus>

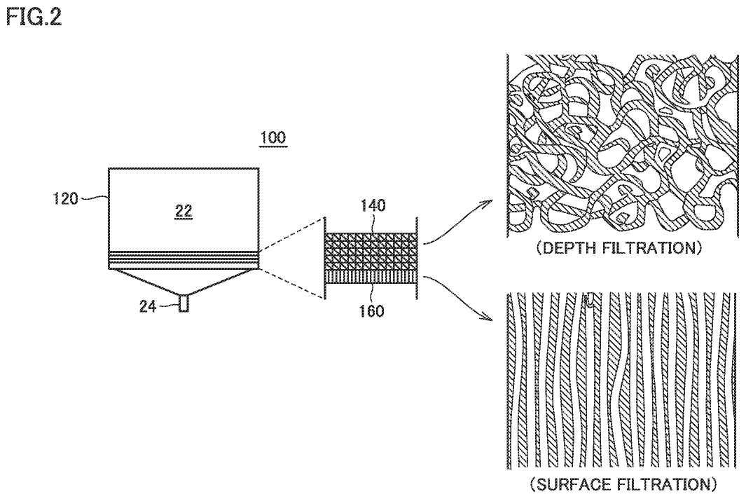

FIG. 2 is a diagram schematically illustrating the configuration of the processing apparatus according to the first embodiment. With reference to FIG. 2, the processing apparatus 100 includes a container 120, a first filter 140, and a second filter 160. The container 120 includes an inner space 22 for housing a liquid sample, and a discharge port 24 provided on the bottom of the container 120.

The first filter 140 and the second filter 160 are provided inside the container 120, and specifically on the bottom of the container 120. The second filter 160 is provided on the downstream of the first filter 140 (closer to the discharge port 24). The first filter 140 is stacked on (the upstream of) the second filter 160. Therefore, when sucked from the discharge port 24, the first filter 140 is in close contact with the second filter 160.

The first filter 140 is configured to permeate blood cells and components smaller than the blood cells. When a liquid sample such as whole blood containing blood cells and measurement objects smaller than the blood cells is filtered by the first filter 140, the measurement objects pass through the first filter 140 faster than the blood cells. At least a part of the measurement objects may pass through the first filter 140 faster than the blood cells, or a part of the blood cells may pass through the first filter 140 faster than a part of the measurement objects.

The first filter 140 may be a filter for surface filtration or a filter for depth filtration as long as the first filter 140 is capable of permeating blood cells and components smaller than the blood cells and advancing the measurement objects to faster than the blood cells.

The first filter 140 includes a mechanism that three-dimensionally and temporarily captures blood cells. As an example of a filter including a mechanism that temporarily captures blood cells, a filter for depth filtration as illustrated in FIG. 2 may be given. Even though the first filter 140 may capture three-dimensionally and temporarily the blood cells, the first filter 140 has a path having a diameter sufficient to allow the blood cells to pass through when a pressure is continuously applied to the blood cells.

The components of blood cells are mainly composed of white blood cells and red blood cells. White blood cells are relatively large particles having a particle size of about 10 to 15 μm. Red blood cells have a particle size of about 7 to 8 μm. The number of red blood cells is significantly greater than that of white blood cells in blood. Therefore, it is preferable that the path of the first filter 140 has a diameter such that at least the red blood cells are allowed to pass through while being captured three-dimensionally and temporarily. Specifically, the first filter 140 has a path having a diameter of at least 7 μm. From another viewpoint, the first filter 140 has a particle retention capacity of 2.7 μm.

It should be noted that the first filter 140 may have a path having a diameter such that platelets having a particle size of about 2 μm contained in blood may be captured three-dimensionally and temporarily.

The filter for depth filtration may be, for example, a depth filter obtained by pressing a fibrous material or a porous membrane having a porous structure.

The first filter 140 may be made of any material such as glass, resin, metal, or ceramics. Considering the fact that blood cells are adsorbed in the path and thereby the path may be blocked by the adsorbed blood cells, the first filter 140 may be made of such a material that the adsorbed blood cells may be desorbed therefrom. For example, the first filter 140 is a glass fiber filter or a cellulose filter.

The second filter 160 selectively captures blood cells by size and selectively permeates the measurement objects by size. Specifically, the second filter 160 have a pore size smaller than the blood cells and larger than the measurement objects. For example, the second filter 160 is configured to have a pore size capable of removing at least those components equal to or larger than red blood cells in the blood cells, and the pore size may be, for example, 2 μm or more and 6 μm or less. The expression that “the blood cells are selectively captured by size and the measurement objects are selectively permeated by size” means that the blood cells are captured in the pores of the filter material, whereas the measurement objects are permeated without being captured in the pores of the filter material. Alternatively, the measurement objects may be temporarily captured when the measurement objects are made to move straightly by the inertial force and collide with the filter material.

The second filter 160 may be a filter for surface filtration or a filter for depth filtration as long as it can remove blood cells. When a membrane filter for surface filtration is used as the second filter 160, it is possible to reliably remove blood cells as compared with the case where a filter for depth filtration is used, which makes it possible to reduce the possibility that the blood cells are mixed in the filtrate.

As an example material of the second filter 160, polyethersulfone, cellulose mixed ester, cellulose acetate, nitrocellulose, polytetrafluoroethylene (PTFE), polyvinylidene fluoride (PVDF), polycarbonate or the like may be given.

<Processing Method>

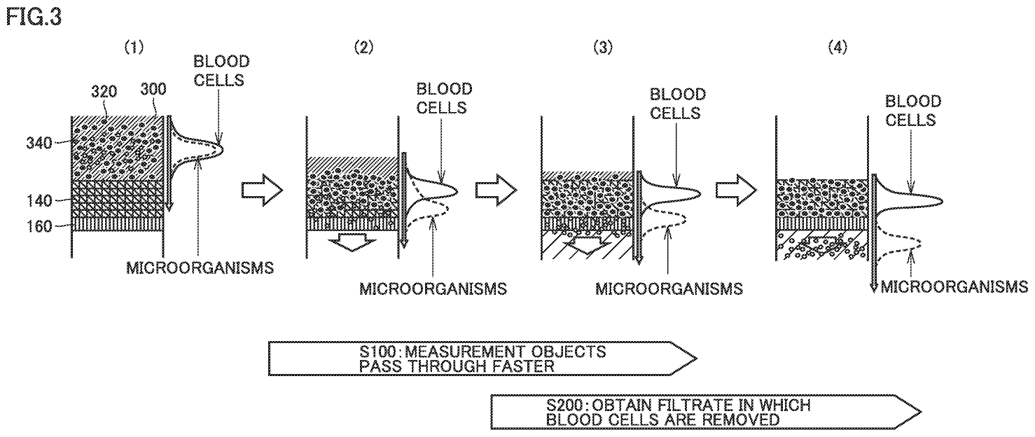

A liquid sample processing method using the processing apparatus will be described with reference to FIG. 3. FIG. 3 is a diagram schematically illustrating the movement of blood cells and measurement objects when a liquid sample is filtered by a first filter and a second filter in the processing apparatus according to the first embodiment.

In FIG. 3, it is assumed that a liquid sample 300 which contains blood cells 320 and microorganisms 340 as the measurement objects is filtered. The size of the microorganisms 340 is smaller than that of the blood cells 320. If the microorganisms are bacteria, the size of the microorganisms 340 is about 1 μm.

The liquid sample 300 containing the blood cells 320 and the microorganisms 340 is, for example, blood collected from a patient and cultured thereafter. Generally, about 10 ml to 30 ml of blood is collected from a patient for culture. In order to recover the measurement objects at a high concentration and high purity, the cultured blood of about 10 ml to 30 ml is preferably processed by the recovery apparatus 1 without being diluted.

Black circles in FIG. 3 indicate the blood cells 320, white circles in FIG. 3 indicate the microorganisms 340, and oblique lines in FIG. 3 indicate plasma (liquid components). In FIG. 3, some reference numerals are omitted. The graph in FIG. 3 illustrates an example concentration distribution in the thickness direction of the membrane (the first filter 140 and the second filter 160). The solid line in the graph illustrates the concentration distribution of blood cells, and the broken line in the graph illustrates the concentration distribution of microorganisms.

Phase (1) in FIG. 3 illustrates a state before filtration, phase (2) and phase (3) in FIG. 3 illustrate states during filtration, and phase (4) in FIG. 3 illustrates a state after filtration. Specifically, when the liquid sample 300 is filtered by the processing apparatus 100, the state of the liquid sample inside the processing apparatus 100 changes over time in the order of phase (1) to phase (4) as illustrated in FIG. 3.

As illustrated in phase (1) of FIG. 3, before filtration, both the blood cells 320 and the microorganisms 340 are dispersed in the liquid sample 300.

When the liquid sample 300 is filtered by suction, the liquid sample 300 first passes through the first filter 140. At this time, the blood cells 320 collide with the filter material in the path of the first filter 140 and are temporarily captured. In contrast, the microorganisms 340 smaller than the blood cells 320 are less likely to be temporarily captured in the path of the first filter 140 than the blood cells 320. Therefore, in the first filtration using the first filter 140, the filtration resistance against the microorganisms 340 is smaller than the filtration resistance against the blood cells 320. In other words, the filtration resistance of the first filter 140 against the microorganisms 340 is smaller than the filtration resistance of the first filter 140 against the blood cells 320. As a result, as illustrated in phase (2) and phase (3) of FIG. 3, as the time elapses, the blood cells 320 and the microorganisms 340 in the liquid sample 300 are gradually separated from each other, and the microorganisms 340 generally pass through the first filter faster than the blood cells 320 in the moving direction of the liquid sample 300.

As illustrated in phase (3) of FIG. 3, since the first filter 140 is stacked on the second filter 160, and after the liquid sample 300 is filtered by the first filter 140, the microorganisms 340 generally pass through the first filter faster than the blood cells 320 in the moving direction of the liquid sample 300, and thereby, the microorganisms 340 reach the second filter 160 earlier than the blood cells 320.

Since the microorganisms 340 generally pass through the first filter faster than the blood cells 320 in the moving direction of the liquid sample 300, the blood cells 320 are retained on the first filter 140, and the liquid sample 300 is subjected to the second filtration using the second filter 160 so as to selectively permeate the microorganisms 340 by size, which makes it possible to obtain a filtrate which contains the microorganisms 340 and from which the blood cells 320 are removed, as illustrated in phase (4) of FIG. 3.

As described above, a liquid sample processing method is realized by filtering the liquid sample 300 using the processing apparatus 100 according to the first embodiment. The liquid sample processing method includes a step (S100) of performing a first filtration on the liquid sample 300 by using a first filter 140 which has a filtration resistance against the microorganisms 340 smaller than the filtration resistance against the blood cells 320, the microorganisms 340 generally pass through the first filter faster than the blood cells 320, and a step (S200) of performing a second filtration while the blood cells 320 are retained on the first filter 140 by using a second filter 160 to selectively permeate the microorganisms 340 by size so as to obtain a final filtrate from which the blood cells 320 are removed out of the liquid sample.

When the first filter 140 is a filter for depth filtration such as a depth filter, the first filtration is depth filtration. When the second filter 160 is a filter for surface filtration such as a membrane filter, the second filtration is surface filtration.

When a liquid sample such as blood is filtered by the second filter 160 without using the first filter 140, the blood cells 320 may be accumulated on the second filter 160 and clog the second filter 160 before the microorganisms 340 pass through the second filter 160. Therefore, when a liquid sample such as blood is filtered only by the second filter 160, the recovery efficiency of the microorganisms 340 is low.

According to the first embodiment, the liquid sample 300 is filtered by the first filter 140, which makes it possible for the microorganisms 340 to pass through the first filter faster than the blood cells 320 in the moving direction of the liquid sample 300. Since the microorganisms 340 pass through the first filter faster than the blood cells 320 in the moving direction of the liquid sample 300, when the liquid sample 300 is filtered by the second filter 160, it is possible for the microorganisms 340 to pass through the second filter 160 before the blood cells 320 are accumulated on the second filter 160 and clog the second filter 160. As a result, it is possible to remove the blood cells 320 out of the liquid sample 300 and efficiently recover the microorganisms 340.

The thickness of the first filter 140 is designed according to, for example, the difference between the permeation rate of the blood cells 320 passing through the membrane and the permeation rate of the microorganisms 340 passing through the membrane, and may be any thickness as long as the blood cells 320 are accumulated on the second filter 160 after the microorganisms 340 has passed through the second filter 160 so as to separate the blood cells 320 and the microorganisms 340 from each other. For example, the first filter 140 may have a thickness of 1.3 mm or more.

The processing apparatus may be configured such that the blood cells 320 do not reach the second filter 160 or such that all or a part of the blood cells 320 reach the second filter 160 at the time when the full amount of the liquid sample 300 has been filtered. The first filtration may be conducted on the liquid sample 300 by using the first filter 140, whereby the microorganisms 340 generally pass through the first filter faster than the blood cells 320, and thereafter, the microorganisms 340 may be selectively permeated by size in the second filtration, whereby the filtrate from which the blood cells have been removed can be obtained without causing clogging.

### Second Embodiment

A liquid sample processing apparatus according to a second embodiment includes a filter unit for collecting measurement objects.

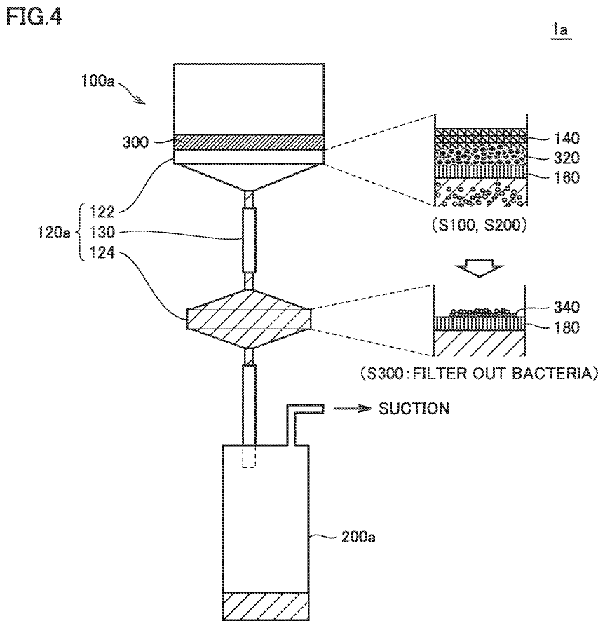

FIG. 4 is a diagram schematically illustrating an overall configuration of a recovery apparatus including a processing apparatus according to a second embodiment. The recovery apparatus 1a according to the second embodiment is different from the recovery apparatus 1 according to the first embodiment in that the recovery apparatus 1a according to the second embodiment is provided with a processing apparatus 100a instead of the processing apparatus 100 and a waste liquid box 200a instead of the suction box 200, and is not provided with the recovery container 220.

The processing apparatus 100a includes a container 120a. The container 120a includes a first filter unit 122, a second filter unit 124, and a connecting pipe 130 that connects the first filter unit 122 and the second filter unit 124.

The first filter unit 122 has the same configuration as the processing apparatus 100 according to the first embodiment. Specifically, the first filter unit 122 is provided with a first filter 140 and a second filter 160. Since the configuration of the first filter unit 122 is the same as that of the processing apparatus 100, the description thereof will not be repeated. Step S100 and step S200 described with reference to FIG. 3 are realized by filtering the liquid sample 300 using the first filter unit 122, and thereby, the filtrate from which the blood cells 320 have been removed is obtained.

The filtrate filtered by the first filter unit 122 is guided to the second filter unit 124 through the connecting pipe 130. A third filter 180 configured to selectively capture the measurement objects by size is disposed in the second filter unit 124. Specifically, the third filter 180 has a pore size smaller than the measurement objects. When the measurement objects are bacteria, the third filter 180 is, for example, a general sterilization filter having a pore size of 0.2 to 0.45 μm.

The third filter 180 is designed in accordance with the size of the measurement objects to be recovered. The third filter 180 may be a filter for surface filtration or a filter for depth filtration as long as it can filter out the measurement objects to be recovered. When a membrane filter for surface filtration is adopted as the third filter 180, the measurement objects are collected on the third filter 180, and on the other hand, when a filter for depth filtration is adopted as the third filter 180, the measurement objects are captured inside the filter. Therefore, when a membrane filter for surface filtration is adopted as the third filter 180, it is expected that the measurement objects may be recovered more easily than the case when a filter for depth filtration is adopted.

After the filtrate is filtered by the first filter unit 122, the filtrate is filtered out by the second filter unit 124, and thereby, the measurement objects can be captured by the third filter 180. The filtrate passed through the second filter unit 124 is recovered in the waste liquid box 200a.

The waste liquid box 200a functions as a waste liquid tank for recovering waste liquid not used in the measurement and also functions as a suction box connected to a vacuum pump.

A liquid sample processing method according to the second embodiment is realized by filtering the liquid sample 300 using the processing apparatus 100a. The liquid sample processing method includes a step (S100) of performing a first filtration on the liquid sample 300 by using a first filter 140 which has a filtration resistance against the microorganisms 340 smaller than the filtration resistance against the blood cells 320 so that the microorganisms 340 generally pass through the first filter faster than the blood cells 320, a step (S200) of performing a second filtration while the blood cells 320 are retained on the first filter 140 by using a second filter 160 to selectively permeate the microorganisms 340 by size so as to obtain a filtrate from which the blood cells 320 are removed out of the liquid sample, and a step (S300) of filtering out the microorganisms 340 from the filtrate.

The processing apparatus 100a according to the second embodiment further includes a third filter 180 disposed on the downstream of the second filter 160 as compared with the processing apparatus 100 according to the first embodiment. Therefore, the measurement objects can be recovered by a single operation of filtering the liquid sample 300 with the processing apparatus 100a, which makes it possible to simply recover the measurement objects.

According to the processing apparatus 100a of the second embodiment, the blood cells 320 are removed by the first filter unit 122, and the microorganisms 340 are recovered by the second filter unit 124. As a result, it is possible to prevent the blood cells 320 from being mixed into the second filter unit 124 at the time of recovering the microorganisms 340. In addition, since the first filter unit 122 and the second filter unit 124 are formed separately, after the liquid sample 300 is filtered, the microorganism recovered by the third filter 180 may be easily collected.

### Third Embodiment

A processing apparatus according to a third embodiment is configured to filter the liquid sample 300 by applying a pressure to an inlet (primary side) instead of the configuration in which the liquid sample 300 is filtered by suction from an outlet (secondary side) where the filtrate is discharged.

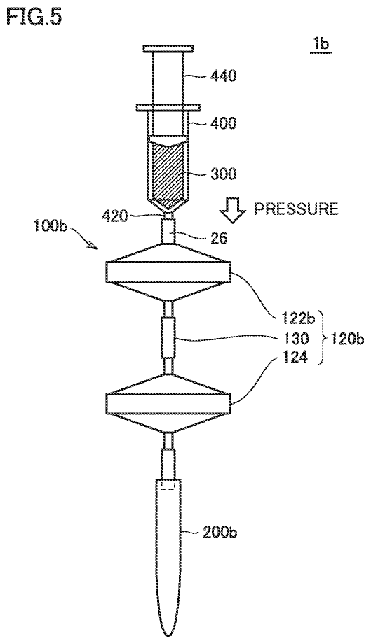

FIG. 5 is a diagram schematically illustrating an overall configuration of a recovery apparatus including a processing apparatus according to a third embodiment. A recovery apparatus 1b according to the third embodiment is different from the recovery apparatus 1a according to the second embodiment in that the recovery apparatus 1b according to the third embodiment is provided with a processing apparatus 100b instead of the processing apparatus 100a and a waste liquid tank 200b instead of the waste liquid box 200a. Unlike the waste liquid box 200a, the waste liquid tank 200b does not function as a suction box. The processing apparatus 100b includes a container 120b instead of the container 120a. The container 120b is substantially the same as container 120a except that the container 120b includes a first filter unit 122b instead of the first filter unit 122. It should be noted that the first filter unit 122b is substantially the same as the first filter unit 122 except that the first filter unit 122b is provided with a connector 26 on the inlet side.

The first filter unit 122b is a syringe filter, and is provided with the connector 26 on the inlet side (primary side) for connection to a cylindrical tip 420 of a syringe 400. After the syringe 400 is filled with the liquid sample 300 and the cylinder tip 420 is connected to the connector 26 of the first filter unit 122b, a pusher 440 is pushed so as to filter the liquid sample 300, and the measurement objects are recovered on the second filter unit 124.

### Modification

Although the liquid sample 300 is filtered by applying a pressure in the first embodiment to the third embodiment, the liquid sample 300 may be filtered without applying any pressure. Alternatively, the first filter 140 and the second filter 160 may be disposed in a centrifugal filtration tube, and the liquid sample 300 may be filtered by applying a centrifugal force to the tube. However, due to the risk that the liquid sample may scatter in the centrifuge, it is preferable that the filtration according to the first embodiment or the second embodiment is conducted by suction filtration or the filtration according to the third embodiment is conducted by using a syringe (pressure filtration). The liquid sample 300 may be filtered by a combination of the pressure filtration and the suction filtration. Specifically, the step of removing the blood cells so as to obtain a final filtrate may be realized by applying a pressure to the first filter 140 from the primary side thereof in the first filtration, or by reducing the pressure inside the second filter 160 from the secondary side thereof in the second filtration, or both. The step of removing the blood cells so as to obtain a final filtrate and the step of filtering out the measurement objects may be realized by applying a pressure, or by reducing a pressure, or both.

In the first embodiment to the third embodiment, the first filter 140 is stacked on the second filter 160, but the arrangement is not limited thereto. For example, the first filter 140 and the second filter 160 may be arranged in close contact with each other in the container 120. Alternatively, the first filter 140 and the second filter 160 may be integrally formed. As long as the microorganisms 340 may pass through the second filter 160 before clogging occurs due to the accumulation of the blood cells 320 on the second filter 160, the first filter 140 may not be stacked on the second filter 160 or a gap may be provided between the first filter 140 and the second filter 160.

### Aspects

It will be appreciated by those skilled in the art that the embodiments and modifications thereof described above are specific examples of the following aspects.

(Clause 1) A liquid sample processing method according to an aspect of the present disclosure includes performing, by using a first filter, a first filtration on a liquid sample, the liquid sample containing blood cells and measurement objects smaller than the blood cells, the first filter having a first filtration resistance against the measurement objects and a second filtration resistance against the blood cells, and the first filtration resistance being smaller than the second filtration resistance, so that the measurement objects generally pass through the first filter faster than the blood cells, and performing, by using a second filter, a second filtration on a filtrate from the first filter, the filtrate containing the measurement objects, while the blood cells are retained on the first filter, to selectively permeate the measurement objects by size so as to obtain a final filtrate in which the blood cells are removed out of the liquid sample.

According to such a configuration, since the second filtration is conducted to permeate the measurement objects while the blood cells are retained on the first filter, it is possible to prevent the clogging caused by the blood cells, which makes it possible to remove the blood cells by a simple filtration.

(Clause 2) In the liquid sample processing method according to clause 1, in the first filtration, the liquid sample is filtered by depth filtration.

According to such a configuration, since the blood cells are captured internally, the movement of the blood cells becomes slower than that of the measurement objects, and as a result, the measurement objects generally pass through the first filter faster than the blood cells.

(Clause 3) In the liquid sample processing method according to clause 1 or 2, in the second filtration, the filtrate containing the measurement objects is filtered by surface filtration.

According to such a configuration, it is possible to reliably remove the blood cells as compared with the depth filtration.

(Clause 4) The liquid sample processing method according to any one of clauses 1 to 3 may further include filtering out the measurement objects from the final filtrate obtained after the second filtration.

According to such a configuration, it is possible to recover the measurement objects from which the blood cells are removed, which makes it possible to purify the measurement objects.

(Clause 5) In the liquid sample processing method according to any one of clauses 1 to 4, the measurement objects include microorganisms.

According to such a configuration, it is possible to purify microorganisms from a liquid sample containing blood cells. In addition, since the second filtration is conducted to permeate the microorganisms while the blood cells are retained on the first filter, it is possible to permeate the microorganisms without being clogged by the blood cells so as to efficiently leave the microorganism in the filtrate. Therefore, it is possible to efficiently recover the microorganisms, and as a result, it is possible to shorten the time required for culturing the recovered microorganisms to a predetermined number.

(Clause 6) In the liquid sample processing method according to any one of clauses 1 to 5, the first filtration and the second filtration is performed by applying a pressure to the first filter from the primary side thereof, or by reducing a pressure inside the second filter from the secondary side thereof, or both.

(Clause 7) A liquid sample processing apparatus according to one aspect of the present disclosure includes a container which houses a liquid sample containing blood cells, a first filter which is disposed in the container, and a second filter which is disposed in the container on the downstream of the first filter. The first filter is configured to permeate the blood cells and measurement objects smaller than the blood cells and allow the measurement objects to permeate faster than the blood cells. The second filter is configured to selectively capture the blood cells by size and selectively permeate the measurement objects by size.

According to such a configuration, since the first filter is configured to allow the measurement objects to permeate faster than the blood cells, the measurement objects reach the second filter disposed on the downstream of the first filter earlier than the blood cells, and since the measurement objects pass through the second filter faster than the blood cells, it is possible to prevent the clogging caused by the blood cells, which makes it possible to remove the blood cells by a simple filtration.

(Clause 8) In the liquid sample processing apparatus according to clause 7, the first filter may be stacked on the upstream of the second filter.

(Clause 9) In the liquid sample processing apparatus according to clause 7 or 8, the first filter may include a mechanism that temporarily captures blood cells.

According to such a configuration, since the blood cells are captured internally, the movement of the blood cells becomes slower than that of the measurement objects, and as a result, the measurement objects pass through the first filter faster than the blood cells.

(Clause 10) In the liquid sample processing apparatus according to clause 9, the first filter may be a depth filter.

(Clause 11) In the liquid sample processing apparatus according to clause 10, the depth filter may be a glass fiber filter.

(Clause 12) In the liquid sample processing apparatus according to any one of clauses 7 to 11, the second filter may have a pore size smaller than the blood cells and larger than the measurement objects.

(Clause 13) In the liquid sample processing apparatus according to any one of clauses 7 to 12, the second filter may be a membrane filter.

According to such a configuration, it is possible to reliably remove the blood cells as compared with a depth filter.

(Clause 14) In the liquid sample processing apparatus according to any one of items 7 to 13, the second filter may have a pore size of 2 μm or more and 6 μm or less.

(Clause 15) The liquid sample processing apparatus according to any one of clauses 7 to 14 may further include a third filter which is disposed in the container on the downstream of the second filter and configured to selectively capture the measurement objects by size.

According to such a configuration, it is possible to recover the measurement objects from which the blood cells are removed, which makes it possible to purify the measurement objects.

(Clause 16) In the liquid sample processing apparatus according to Clause 15, the container is provided with a first filter unit which includes the first filter and the second filter, and a second filter unit which includes the third filter and is disposed on the downstream of the first filter unit.

According to such a configuration, the blood cells are removed by the first filter, and the measurement objects are recovered in the second filter. As a result, it is possible to prevent blood cells from being mixed into the second filter when recovering the measurement objects.

(Clause 17) In the liquid sample processing apparatus according to any one of clauses 7 to 16, the measurement objects include microorganisms.

According to such a configuration, it is possible to purify microorganisms from a liquid sample containing blood cells. In addition, since the second filtration is conducted to permeate the microorganisms while the blood cells are retained on the first filter, it is possible to permeate the microorganisms without being clogged by the blood cells so as to efficiently leave the microorganism in the filtrate. Therefore, it is possible to efficiently recover the microorganisms, and as a result, it is possible to shorten the time required for culturing the recovered microorganisms to a predetermined number.

Although the embodiments of the present disclosure have been described, it should be understood that the embodiments disclosed herein are illustrative and not restrictive in all respects. The scope of the present disclosure is indicated by the claims, and all modifications within the meaning and scope equivalent to the claims are intended to be included.

What is claimed is:

1. A liquid sample processing method comprising:
performing, by using a first filter, a first filtration on a liquid sample, the liquid sample containing blood cells and measurement objects smaller than the blood cells, the first filter having a first filtration resistance against the measurement objects and a second filtration resistance against the blood cells, and the first filtration resistance being smaller than the second filtration resistance, so that the measurement objects generally pass through faster than the blood cells; and
performing, by using a second filter, a second filtration on a filtrate from the first filter, the filtrate containing the measurement objects, while the blood cells are retained on the first filter, to selectively permeate the measurement objects by size so as to obtain a final filtrate in which the blood cells are removed out of the liquid sample.

2. The liquid sample processing method according to claim 1, wherein
in the first filtration, the liquid sample is filtered by depth filtration.

3. The liquid sample processing method according to claim 1, wherein
in the second filtration, the filtrate containing the measurement objects is filtered by surface filtration.

4. The liquid sample processing method according to claim 1, further comprising:
filtering out the measurement objects from the final filtrate obtained after the second filtration.

5. The liquid sample processing method according to claim 1, wherein
the measurement objects are microorganisms.

6. The liquid sample processing method according to claim 1, wherein
the first filtration and the second filtration is performed by applying a pressure to the first filter from the primary side thereof, or by reducing a pressure inside the second filter from the secondary side thereof, or both.

7. A liquid sample processing apparatus comprising:
a container which houses a liquid sample containing blood cells;
a first filter which is disposed in the container and configured to permeate the blood cells and measurement objects smaller than the blood cells and allow the measurement objects to permeate faster than the blood cells; and
a second filter which is disposed in the container on the downstream of the first filter and configured to selectively capture the blood cells by size and selectively permeate the measurement objects by size.

8. The liquid sample processing apparatus according to claim 7, wherein
the first filter is stacked on the upstream of the second filter.

9. The liquid sample processing apparatus according to claim 7, wherein
the first filter includes a mechanism that temporarily captures the blood cells.

10. The liquid sample processing apparatus according to claim 9, wherein
the first filter is a depth filter.

11. The liquid sample processing apparatus according to claim 10, wherein
the depth filter is a glass fiber filter.

12. The liquid sample processing apparatus according to claim 7, wherein
the second filter has a pore size smaller than the blood cells and larger than the measurement objects.

13. The liquid sample processing apparatus according to claim 7, wherein
the second filter is a membrane filter.

14. The liquid sample processing apparatus according to claim 7, wherein
the second filter has a pore size of 2 μm or more and 6 μm or less.

15. The liquid sample processing apparatus according to claim 7 further comprising:
a third filter which is disposed in the container on the downstream of the second filter and configured to selectively capture the measurement objects by size.

16. The liquid sample processing apparatus according to claim 15, wherein
the container is provided with a first filter unit which includes the first filter and the second filter, and a second filter unit which includes the third filter and is disposed on the downstream of the first filter unit.

17. The liquid sample processing apparatus according to claim 7, wherein
the measurement objects are microorganisms.
